(19) United States
(12) Patent Application Publication
(10) Pub. No.: US 2021/0330914 A1
(43) Pub. Date: Oct. 28, 2021

(54) CONTROLLING THE OPERATION OF A VENTILATOR

(71) Applicant: SPARKCOGNITION, INC., Austin, TX (US)

(72) Inventors: JOSH YOUNG, AUSTIN, TX (US); GUILLAUME HERVE, AUSTIN, TX (US); TRAVIS F. MEITZEN, AUSTIN, TX (US); MILTON LOPEZ, ROUND ROCK, TX (US)

(21) Appl. No.: 17/239,287

(22) Filed: Apr. 23, 2021

Related U.S. Application Data
(60) Provisional application No. 63/014,678, filed on Apr. 23, 2020.

(51) Int. Cl. A61M 16/00; G06N 3/08; G05B 13/04; G05B 13/02; G16H 40/63; A61M 16/16

(52) U.S. Cl. CPC A61M 16/024; G06N 3/088; G05B 13/042; G05B 13/027; G16H 40/63; A61M 16/0078; A61M 2205/8206; A61M 2016/0027; A61M 2230/205; A61M 2016/003; A61M 2205/3368; A61M 2205/52; A61M 2205/18; A61M 16/161

(57) ABSTRACT

A ventilator, comprising: a manual resuscitator; a motor; a compression plate that is mechanically coupled to the motor, wherein operation of the motor causes the compression plate to periodically compress the manual resuscitator against a resuscitator plate; and one or more operational parameter adjustment controls, wherein operation of the motor is adjusted in response to a value of a particular operational parameter being changed using the one or more operational parameter adjustment controls.

### CROSS-REFERENCE TO RELATED APPLICATIONS

This is a Non-Provisional application for patent claiming the benefit of U.S. Provisional Patent Application Ser. No. 63/014,678, filed Apr. 23, 2020, the contents of which are hereby incorporated by reference in its entirety.

### BRIEF DESCRIPTION OF DRAWINGS

FIG. 1 sets forth a first perspective of internal components of a ventilator in accordance with some embodiments of the present disclosure.

FIG. 2 sets forth a second perspective of internal components of a ventilator in accordance with some embodiments of the present disclosure.

FIG. 3 sets forth a diagram of a ventilator in accordance with some embodiments of the present disclosure.

FIG. 4A sets forth a flow chart illustrating an example method of operating a ventilator 400 in accordance with some embodiments of the present disclosure.

FIG. 4B sets forth a flow chart illustrating an additional example method of operating a ventilator 400 in accordance with some embodiments of the present disclosure.

FIG. 5 is a diagram of an example of a system to generate one or more trained models that are used in conjunction with controlling a ventilator in accordance with some examples of the present disclosure.

FIG. 6A and FIG. 6B collectively illustrate an example of a method of cooperative execution of a genetic algorithm and an optimization trainer to generate an autoencoder in accordance with some embodiments of the present disclosure.

FIG. 7 illustrate an example of a system for using an autoencoder to monitor one or more devices for operational anomalies in accordance with some embodiments of the present disclosure.

### DESCRIPTION OF EMBODIMENTS

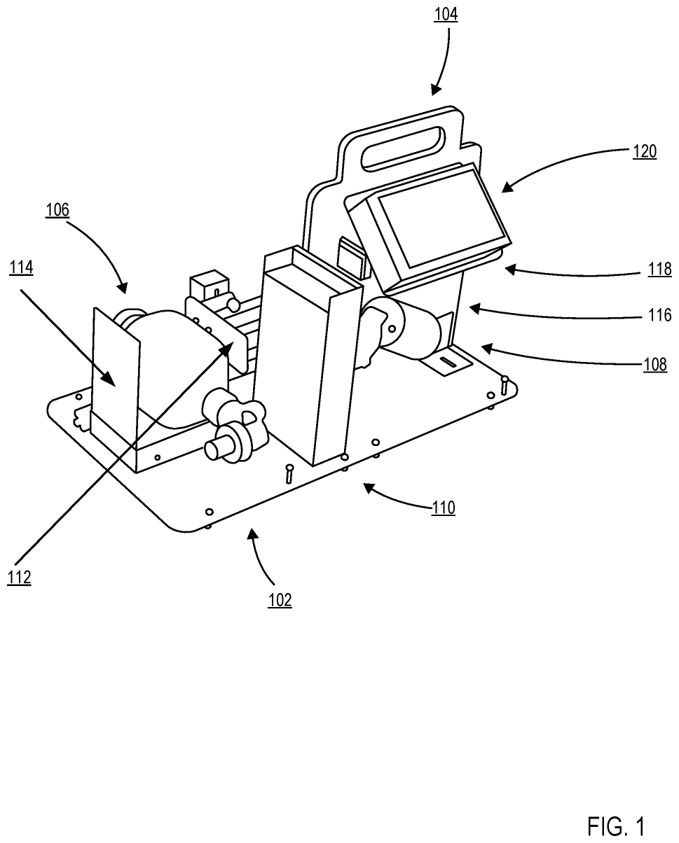

Example ventilators, methods for controlling the operation of a ventilator, and products for controlling the operation of a ventilator in accordance with some embodiments of the present disclosure are described with reference to the accompanying drawings, beginning with FIG. 1. FIG. 1 sets forth a first perspective of internal components of a ventilator 100 in accordance with some embodiments of the present disclosure. Readers will appreciate that in order to illustrate the internal components of the ventilator 100, some components of ventilators in accordance with some embodiments of the present disclosure are omitted from FIG. 1. In some embodiments, however, the ventilator 100 may also include a housing, one or more transparent portions of a housing, a handle, user interfaces, interfaces to some of the components depicted in FIG. 1, and other components.

The ventilator 100 depicted in FIG. 1 includes a plurality of components that are depicted in this example as being mounted on a horizontal mount plate 102 and a plurality of components that are depicted in this example as being mounted on a vertical mount plate 104. For example, the ventilator depicted in FIG. 1 includes a manual resuscitator 106 and a motor 108. The manual resuscitator 106 depicted in FIG. 1 may be embodied, for example, as a bag valve mask (BVM), sometimes known by the proprietary name Ambu bag. The manual resuscitator 106 may be a device that is often used to provide positive pressure ventilation to patients who are not breathing (or not breathing adequately). In some embodiments, the manual resuscitator 106 may include a flexible air chamber (i.e., the bag) that can be attached to an attached to a face mask via a shutter valve, such that breathing assistance may be provided to a patent that is wearing the face mask by compressing the flexible air chamber, which reinflates when the bag is not being compressed. In some embodiments, the manual resuscitator 106 can deliver breathing assistance to a patient without the use of a face mask, as the manual resuscitator 106 may utilize an alternative airway adjunct such as, for example, an endotracheal tube or a laryngeal mask airway.

The motor 108 depicted in FIG. 1 may be embodied, for example, as an electric motor that converts electrical energy into mechanical energy. For example, the motor 108 may be embodied as a windshield wiper motor that runs on 12V of electricity, which may be provided by the power supply 110 depicted in FIG. 1.

In the example depicted in FIG. 1, the motor 108 may be mechanically coupled to a compression plate 112. The motor 108 may be mechanically coupled to a compression plate 112, for example, through the use of one or more movable linkages. In such an example, operation of the motor 108 may cause the compression plate 112 to move via the linkages such that the compression plate 112 operates as a piston that moves back and forth along an axis. In such an example, due the positioning of the compression plate 112, the manual resuscitator 106, and a fixed resuscitator plate 114, operating the motor may cause the compression plate 112 to periodically compress the manual resuscitator 106 against a resuscitator plate 114. As described above, through compression (when the compression plate 112 is positioned so as to compress the manual resuscitator 106 against a resuscitator plate 114) and reinflation of the manual resuscitator 106 (when the compression plate 112 is positioned so as to not compress the manual resuscitator 106 against a resuscitator plate 114), breathing assistance may be delivered to a patient.

The ventilator 100 depicted in FIG. 1 also includes a motor controller 116 for controlling the operation of the motor 108, as will as one of more computer processors 118 and a user interface 120. The motor controller 116 and the one of more computer processors 118 may be part of a larger ventilator control system that may include computer program instructions (referred to herein as a motor control model) that may be executed on the one of more computer processors 118. The motor control model may include, for example, one or more modules of computer program instructions that, when executed, can impact the extent to which power is delivered to the motor 108 from a power supply 110. The motor control model may be configured, for example, to influence the manner in which power is delivered to the motor 108 to improve the function or longevity of ventilator, to influence the manner in which power is delivered to the motor 108 to deliver improved breathing assistance to a patient, and so on. The motor control model, including the creation thereof through machine learning techniques, will be described in greater detail below.

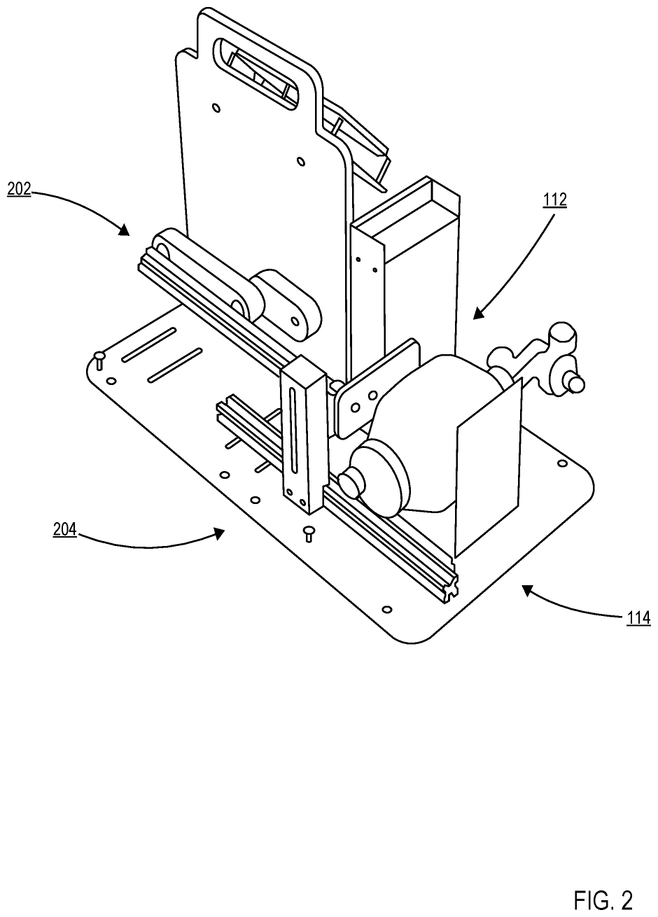

For further explanation, FIG. 2 sets forth a second perspective of internal components of a ventilator 100 in accordance with some embodiments of the present disclosure. The perspective depicted in FIG. 2 includes an illustration of the linkage system 202 that is used to couple the motor 108 to the compression plate 112. The linkage system 202 may be embodied, for example, as an assembly of bodies connected to manage forces and movement of the compression plate 112. In such an example, as the motor 108, the motor generates a torque that is used to move the linkage system 202. Because the compression plate 112 is fixed to one or more components of the linkage system 202, moving the linkage system 202 causes the compression plate 112.

In the example depicted in FIG. 2, the ventilator 100 also includes a linkage guide and mount 204 that controls the direction that the linkage system 202 and the compression plate 112 are moved. When the compression plate 112 is moved towards the fixed resuscitator plate 114, the compression plate 112 exerts a force on the bag of the manual resuscitator 106, thereby causing compression of the manual resuscitator 106. When the compression plate 112 is moved away from the fixed resuscitator plate 114, the compression plate 112 exerts less force on the bag of the manual resuscitator 106, thereby enabling the bag to reinflate.

In some embodiments, ventilators may include additional components beyond those that are depicted in FIG. 1 and FIG. 2. For example, ventilators in accordance with some embodiments of the present disclosure may include one or more operational parameter adjustment controls. The one or more operational parameter adjustment controls may be embodied, for example, as a mechanical device such as a button, a knob, or other device that enables an operator of the ventilator to adjust one or more operating parameters associated with the ventilator. The one or more operational parameter adjustment controls may be used to set or change the operating mode for the ventilator, to set or change the number of compressions of per unit of time of the manual resuscitator 106 that should occur, to set or change the inspiratory to expiratory ratio for the manual resuscitator 106, to set or change the tidal volume for the manual resuscitator 106, and others. In other embodiments, the one or more operational parameter adjustment controls may be embodied in other ways such as through the use of icons on a touchscreen display. Readers will appreciate that the operation of the motor 108 may be adjusted in response to a value of a particular operational parameter being changed using the one or more operational parameter adjustment controls. For example, the operation of the motor 108 may be adjusted to perform a different number of cycles per unit of time, the operation of the motor 108 may be adjusted to deliver more torque, and so on.

Ventilators in accordance with some embodiments of the present disclosure may also include one or more sensors. In general, the sensors may be broken up into two broad categories of sensors. A first category of sensors relates to sensors whose outputs can monitor the health of the patient and can be used to control the operation of the ventilator to improve the health of the patient. Such sensors can include, for example, a pressure sensor, a patient pulse oximeter, an inline oxygen flow meter, a mass flow sensor, an electrical sensor, a humidity sensor, a temperature sensor, other sensors, and combinations of such sensors. These sensors may be located on the ventilator itself (or some portion thereof), such as being placed along the airway between the manual resuscitator and the patient's face mask.

A second category of sensors relates to sensors whose outputs can monitor the health of the ventilator and can be used to for predictive maintenance or prescriptive maintenance purposes as described in greater detail below. Through the usage of such sensors and artificial intelligence enabled solutions, ventilators may be created relatively quickly and at a relatively low cost with easily obtained components as compared to traditional ventilators. These artificial intelligence enabled ventilators may operate as reliably or even more reliably than their traditional counterparts, through the pairing of the ventilator with predictive maintenance solutions and other solutions as described in greater detail below.

In some embodiments, the one of more computer processors 118 may be configured to execute computer program instructions that adjust the operation of the ventilator 100 in response to sensor data received from the one or more sensors. For example, a pressure sensor that is located between a face mask worn by a patient and an inspiratory valve of the manual resuscitator 106 may be utilized to capture data, where sensor data that is output by the pressure sensor may be used to detect irregularities with the patient's ventilation and adjust the operation of the ventilator 100 (e.g., by changing the operation of the motor 108) automatically and without user intervention, as will be described in greater detail below.

In other embodiments, the one of more computer processors 118 may be configured to execute computer program instructions that generate alerts in response to sensor data that is received from the one or more sensors. Continuing with the example in which a pressure sensor is located between a face mask worn by a patient and an inspiratory valve of the manual resuscitator 106 may be utilized to capture data, sensor data that is output by the pressure sensor may be used to detect a mechanical malfunction of the ventilator 100. For example, a pressure reading may indicate that there is a leak or blockage in some portion of the ventilator 100, a pressure reading may indicate that the motor 108 has failed, and so on. In response to detecting the occurrence of such an event, alerts may be taken, although in other embodiments corrective actions (e.g., power cycling the motor 108) may be taken.

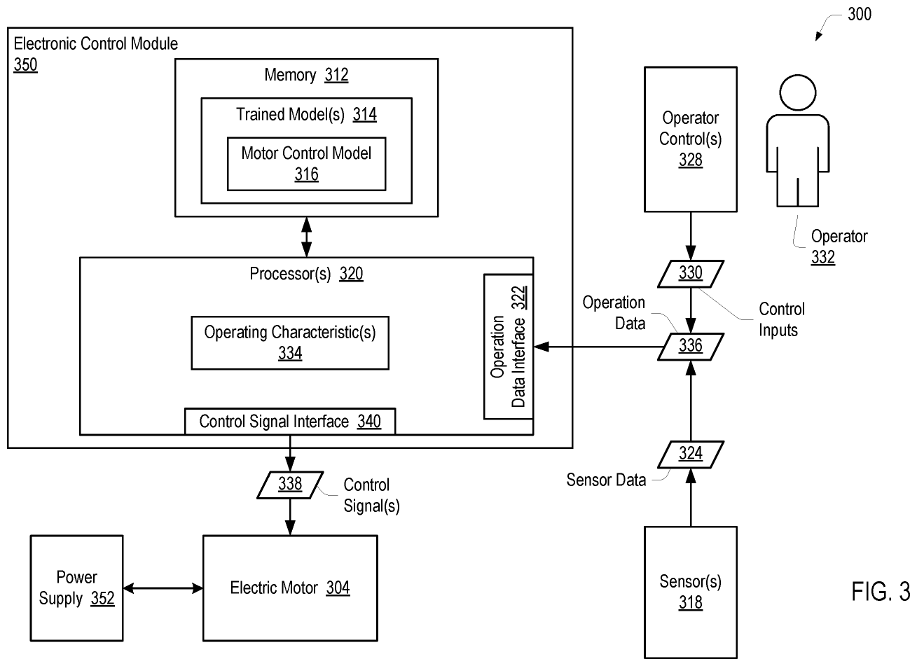

For further explanation, FIG. 3 sets forth a diagram of a ventilator 300 in accordance with some embodiments of the present disclosure. Although different details of the ventilator 300 are illustrated in FIG. 3, the ventilator 300 depicted in FIG. 3 may be similar to the ventilators described above with reference to FIG. 1 and FIG. 2 as the ventilator 300 may also include the components described above. The ventilator 300 depicted in FIG. 3 includes an electric motor 304, a power supply 352, a memory 312, and one or more operator controls 328 (e.g., the operational parameter adjustment controls) that are coupled to one or more processors 320. Readers will appreciate that although only a single power supply 352 is depicted for ease of explanation, additional sources of electrical power (e.g., a backup battery) may actually be included in the ventilator 300.

The ventilator 300 depicted in FIG. 3 also includes one or more sensors 318 configured to generate sensor data 324 that may be used to create operation data 336 for the ventilator 300. The operation data 336 can include sensor data 324 describing the operation of the ventilator 300, sensor data 324 describing the condition of a patient, some other form of sensor data 324, or a combination thereof. The operation data 336 can also include control inputs 330 generated from the operator controls 328, such that the operation data 336 can include a combination of data generated from sensors and data generated in response to an operator 332 (e.g., a nurse, a doctor) interacting with the ventilator 300. Such operation data 336 may be received, for example, through the use of an operation data interface 322.

The ventilator 300 depicted in FIG. 3 also includes a memory 312 that is coupled to one or more processors 320. The one or more processors 320 may include one or more single-core or multi-core central processing units (CPUs), one or more digital signal processors (DSPs), one or more graphics processing units (GPUs), or any combination thereof. In this example, the memory 312 and the one or more processors 320 are incorporated in an electronic control module 350 (“ECM”) that is coupled to the electric motor 304, the sensors 318, and the one or more operator controls 328. In some implementations, the memory 312 includes volatile memory devices, non-volatile memory devices, or both, such as one or more hard drives, solid-state storage devices (e.g., flash memory, magnetic memory, or phase change memory), a random-access memory (RAM), a read-only memory (ROM), one or more other types of storage devices, or any combination thereof. Although the memory 312 and the one or more processors 320 are depicted within the electronic control module 350, in other implementations, one or both of the memory 312 and the one or more processors 320 may be external to the electronic control module 350.

The memory 312 depicted in FIG. 3 includes one or more trained models 314. The one or more trained models 314 (e.g., trained machine learning models) may be executable by the one or more processors 320 to initiate, perform, or control various operations of the ventilator 300. The one or more trained models 314 in FIG. 3 may include, amongst other models, a motor control model 316.

The motor control model 316 may be embodied, for example, as one or more modules of computer program instructions that, when executed, can control the operation of the motor 304. For example, the motor control model 316 may be configured to operate the electric motor 304 in such a way so as to cause a compression plate to periodically compress a manual resuscitator against a resuscitator plate and, responsive to receiving a signal (in the form of the operation data 336), adjust the operation of the electric motor 304. The motor control model 316 may be further configured to control the operation of the motor 304, for example, in order to adjust the operation of the ventilator 300 in response to detecting a possible malfunction with the ventilator 300, in order to adjust the operation of the ventilator 300 to adjust the manner in which the ventilator 300 assists the breathing of a patient, or for other reasons. The motor control model 316 may control the operation of the motor 304 through the use of a control signal interface 340 that can send a control signal 338 to the electric motor 304. The motor control model 316, including the creation thereof through machine learning techniques, will be described in greater detail below.

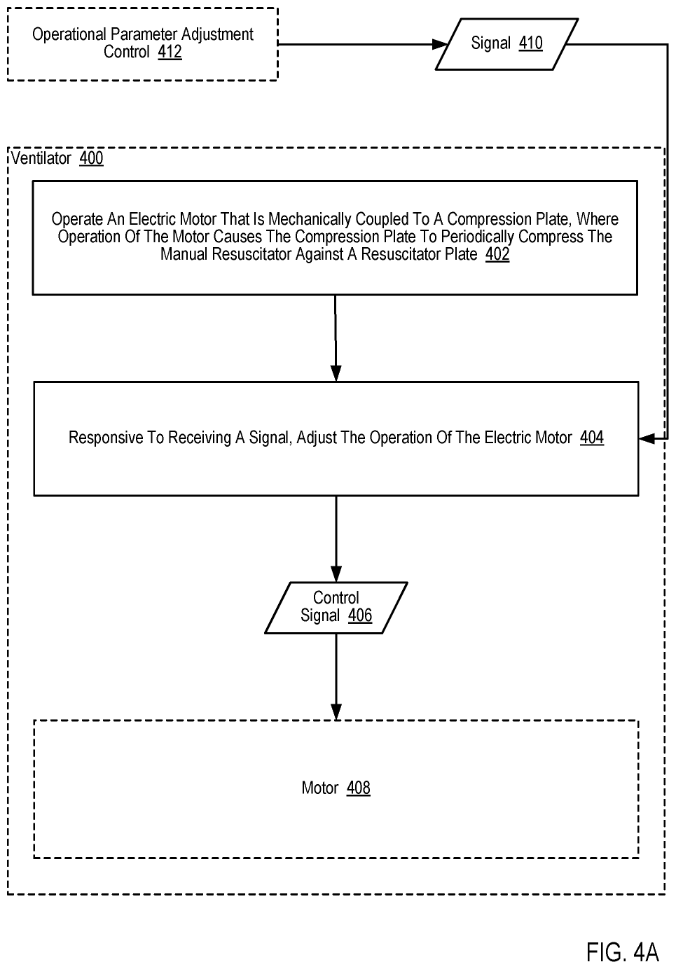

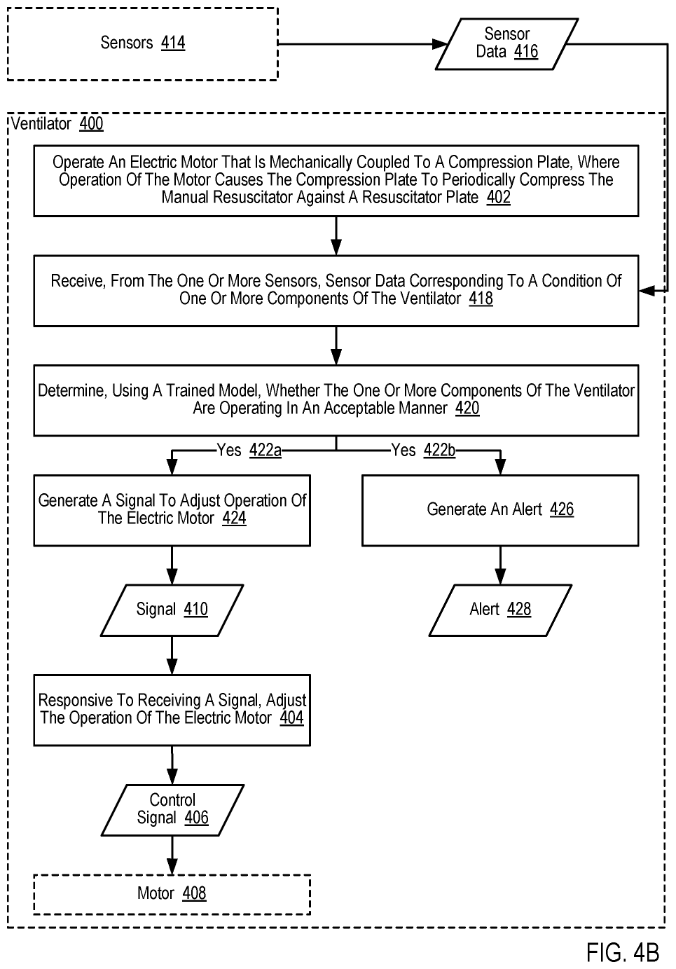

For further explanation, FIG. 4A sets forth a flow chart illustrating an example method of operating a ventilator 400 in accordance with some embodiments of the present disclosure. Although depicted in less detail, the ventilator 400 of FIG. 4A may be similar to the ventilators described elsewhere in the present disclosure and may contain the same, additional, or fewer components described elsewhere in the present disclosure. In fact, the example method depicted in FIG. 4A may be carried out by an apparatus such as an ECM that is included in the ventilator 400 as described above.

The example method depicted in FIG. 4A includes operating 402 an electric motor 408. Operating 402 an electric motor 408 may be carried out, for example, by delivering power to the electric motor through a battery, a power supply, or another power source. Once the electric motor 408 is powered up, a control signal may be sent to the electric motor to begin operation of the electric motor 408. In such an example, the initial operating state of the electric motor (e.g., how many cycles per unit of time that the motor is performing, the amount of torque generated by the motor) may be set in a variety of ways. For example, the electric motor 408 may operate in accordance with its most recently used performance settings, the electric motor 408 may operate in accordance with its default performance settings, or the electric motor 408 may operate in accordance with some other settings, including settings that were determined by a trained model.

Consider an example in which a model was trained using data describing various ventilator related settings that were utilized for patients of a certain age, patients of a certain weight range, patients with certain conditions, and so on. In such an example, information describing how the patients responded to various ventilator related settings may also be utilized to generate a model that can identify optimal ventilator related settings for a particular patient, based on the characteristics of that patient. In such an example, this trained model may be supported by the ECM (or by some other execution environment) such that providing the trained model with information describing a particular patient may cause the trained model to generate, as output, information describing the anticipated best ventilator settings for that patient. In other words, the trained model may be used to determine one or more initial operating parameters for the ventilator based on information describing a patient. For example, if a particular patient was a male between the ages of 48-52 with a body mass index between 18-20, the model may determine (based on analyzing large volumes of data describing patient's historical responses to various ventilator settings), that the ventilator should initially be set to support a respiratory rate of 27 beats per minute with a tidal volume of 525 mL. In such an example, the electric motor may be configured to deliver a particular torque at a particular rate that would cause the manual resuscitator to support a respiratory rate of 27 beats per minute with a tidal volume of 525 mL. Although not depicted in FIG. 4A, the electric motor 408 may be mechanically coupled to a compression plate, wherein operation of the motor causes the compression plate to periodically compress the manual resuscitator against a resuscitator plate, as described above.

The example method depicted in FIG. 4A also includes, responsive to receiving a signal 410, adjusting 404 the operation of the electric motor 408. In the example method depicted in FIG. 4A, the signal 410 may be received in response to a value of a particular operational parameter being changed using the one or more operational parameter adjustment controls 412. Consider an example in which the operational parameter adjustment controls 412 include one or more buttons through which an operator can adjust the tidal volume to be delivered by the ventilator. In such an example, if an operator presses such buttons to change the tidal volume settings associated with the ventilator, the operation of the electric motor 408 can be adjusted 404 so as to cause the compression plate to periodically compress the manual resuscitator against a resuscitator plate with a force that will cause the manual resuscitator to produce a tidal volume that corresponds to the adjusted setting. In other embodiments, the signal 410 may be generated in response to the analysis of sensor data by a trained model, or in some other way. In such an example, adjusting 404 the operation of the electric motor 408 may be carried out through the use of a control signal 406 as described in other portions of the present disclosure.

For further explanation, FIG. 4B sets forth a flow chart illustrating an additional example method of operating a ventilator 400 in accordance with some embodiments of the present disclosure. Although depicted in less detail, the ventilator 400 of FIG. 4B may be similar to the ventilators described elsewhere in the present disclosure and may contain the same, additional, or fewer components described elsewhere in the present disclosure. In fact, the example method depicted in FIG. 4B may be carried out by an apparatus such as an ECM that is included in the ventilator 400 as described above. The example method depicted in FIG. 4B is similar to the example method depicted in FIG. 4A, as the example method depicted in FIG. 4B also includes operating 402 an electric motor 408 and, responsive to receiving a signal 410, adjusting 404 the operation of the electric motor 408.

In the example method depicted in FIG. 4B, the ventilator includes one or more sensors 414. The one or more sensors may include a pressure sensor that detects feedback from patient's breathing, thereby enabling the adjustment of the ventilator 400 to an appropriate pressure to improve a patient's breathing. Likewise, the pressure sensor may be used to detects mechanical malfunction of the ventilator 400 as described above. The one or more sensors may also include a patient pulse oximeter that measures oxygenation level of the patient's blood and pulse rate. If oxygenation levels are too low, for example, operation of the ventilator 400 may be adjusted to improve a patient's oxygenation level. The one or more sensors may also include an inline oxygen flow meter to detect the level of oxygen being supplemented to the patient and, using pulse oximeter readings, throttle air flow as needed. The one or more sensors may also include a mass flow sensor to gather improved breath tracking for data more accurate understanding of patient's breathing cycle, which may in turn be used to adjust the operation of the ventilator 400 to improve a patient's breathing. The one or more sensors may also include an electrical sensor for anticipatory breathing assistance if patient is not in a medically induced coma, a humidity sensor to control a humidifier system, a temperature sensor for air quality and humidifying calculations, and other sensors in accordance with embodiments of the present disclosure. Readers will appreciate that the ventilators described herein may also implement various combinations of the sensors described above and may even package groups of sensors such that ventilators with a variety of designs and complexity may be utilized as needed.

The example method depicted in FIG. 4B also includes receiving 418, from the one or more sensors 414, sensor data 416 corresponding to a condition of one or more components of the ventilator 400. As described above, data from a pressure sensor may be used to indicate that some component of the ventilator 400 is comprised as low-pressure readings may indicate, for example, that the ventilator has a leak, that the compression plate is not compressing the manual resuscitator against a resuscitator plate as expected or may be otherwise indicative of a failure with one or more components of the ventilator 400. In accordance with embodiments of the present disclosure, other sensors may also be used to monitor the condition of one or more components of the ventilator 400. For example, a temperature sensor may be used to monitor the motor 408 and detect that the motor is overheating, a force sensor may be used to verify that a force exerted by the compression plate is as expected, and so on. As such, sensor data 416 may be received and the sensor data may be analyzed by a trained model to determine that the sensor data 416 corresponds to a known condition (e.g., an ‘operating properly’ condition, an ‘operating improperly’ condition, a ‘disabled’ condition, an ‘offline’ condition, a ‘failing’ condition) of one or more components of the ventilator 400. In such an example, this sensor data 416 may be used to evaluate whether one or more components of the ventilator 400 are operating as expected.

The example method depicted in FIG. 4B also includes determining 420, using a trained model, whether the one or more components of the ventilator 400 are operating in an acceptable manner. Determining 420 whether the one or more components of the ventilator 400 are operating in an acceptable manner may be carried out, for example, using a trained model that evaluates sensor data 416 to determine whether components have failed, are about to fail, or otherwise are not operating as expected. In such an example, the model may be trained using historical sensor data to identify components that have failed, are about to fail, or otherwise are not operating as expected. In other embodiments, determining 420 whether the one or more components of the ventilator 400 are operating in an acceptable manner may be carried by a simple comparison between one or more sensor values and one or more specifications, to identify sensors that are recording operational characteristics that are not within expected specifications.

The example method depicted in FIG. 4B also includes, responsive to affirmatively 422a determining that the one or more components of the ventilator 400 are not operating in an acceptable manner, generating 424 a signal 410 to adjust operation of the electric motor 408. Consider an example in which sensor data indicates that the that the tidal volume of being produced by the ventilator is 525 mL but the ventilator 400 is configured so as to produce a tidal volume of 650 mL. In such an example, it may be determined 420 above the bag for the manual resuscitator is not operating as expected based on measurements of the amount of force that is being applied to the bag by the compression plate, as applying such a force would be expected to produce a tidal volume of 650 mL. In such an example, a signal 410 may be generated 424 to adjust the operation of the motor 408 in such a way that the compression plate exerts additional pressure on the bag in an effort to achieve the desired tidal volume.

The example method depicted in FIG. 4B also includes, responsive to affirmatively 422b determining that the one or more components of the ventilator 400 are not operating in an acceptable manner, generating 426 an alert 428. The alert 428 may be generated, for example, for presentation on a display associated with the ventilator 400, to produce a noise to alert medical personal to the problem, to illuminate an indicator light, or in some other way. In some embodiments, the alert 428 may include information describing a projected failure of one or more components of the ventilator 400. The projected failure may be identified through the use of one or more trained models that are configured to identify potential failures based on sensor data or some other data. As such, in order to address a projected failure, the ventilator 400 may be repaired or replaced so as to avoid placing the patient a situation where there are without breathing assistance.

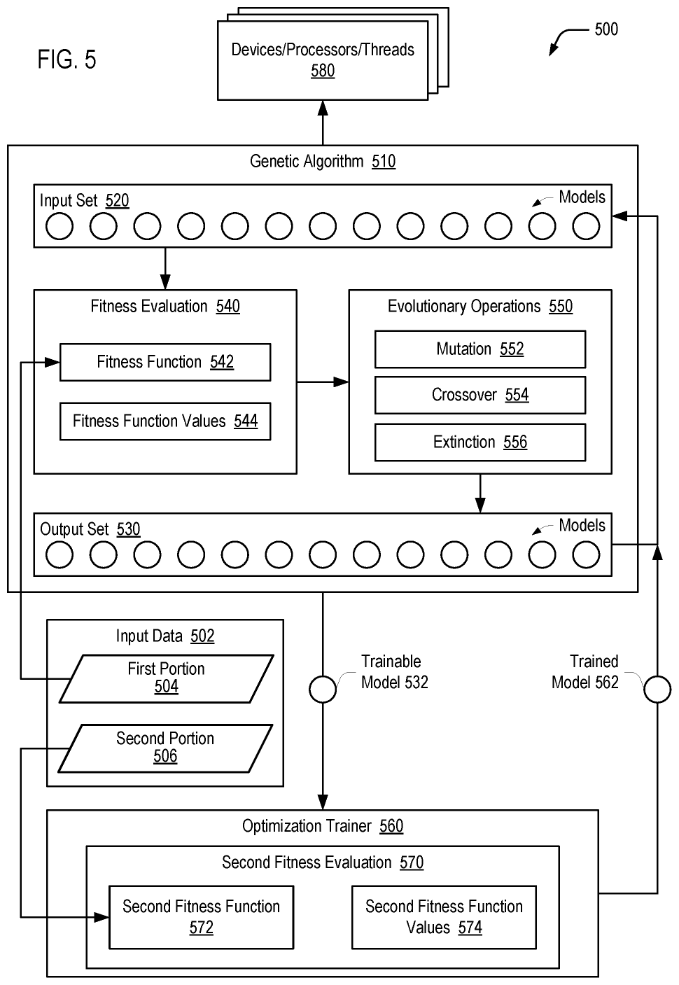

For further explanation, FIG. 5 sets forth an example of a system 500 for generating a machine learning data model, such as one or more of the trained models that can be used by the one or more processors, the ECM, or the ventilator as described above. Although FIG. 5 depicts a particular example for purpose of explanation, in other implementations other systems may be used for generating or updating one or more of the trained models.

In some examples, the system 500, or portions thereof, may be implemented using (e.g., executed by) one or more computing devices, such as laptop computers, desktop computers, mobile devices, servers, and Internet of Things devices and other devices utilizing embedded processors and firmware or operating systems, etc. In the illustrated example, the system 500 includes a genetic algorithm 510 and an optimization trainer 560. The optimization trainer 560 is, for example, a backpropagation trainer, a derivative free optimizer (DFO), an extreme learning machine (ELM), etc. In particular implementations, the genetic algorithm 510 is executed on a different device, processor (e.g., central processor unit (CPU), graphics processing unit (GPU) or other type of processor), processor core, and/or thread (e.g., hardware or software thread) than the optimization trainer 560. The genetic algorithm 510 and the optimization trainer 560 are executed cooperatively to automatically generate a machine learning data model (e.g., one of the trained models described above and referred to herein as “models” for ease of reference), such as a neural network or an autoencoder, based on the input data 502. In this example, the system 500 performs an automated model building process that enables users, including inexperienced users, to quickly and easily build highly accurate models based on a specified data set.

In some implementations, during configuration of the system 500, a user specifies the input data 502. In some implementations, the user may also specify one or more characteristics of models that can be generated. In such implementations, the system 500 constrains models processed by the genetic algorithm 510 to those that have the one or more specified characteristics. For example, the specified characteristics may constrain allowed model topologies (e.g., to include no more than a specified number of input nodes or output nodes, no more than a specified number of hidden layers, no recurrent loops, etc.). In some examples, constraining the characteristics of the models can reduce the computing resources (e.g., time, memory, processor cycles, etc.) needed to converge to a final model, can reduce the computing resources needed to use the model (e.g., by simplifying the model), or both.

In some implementations, a user can configure aspects of the genetic algorithm 510 via input to graphical user interfaces (GUIs). For example, the user may provide input to limit a number of epochs that will be executed by the genetic algorithm 510. Alternatively, the user may specify a time limit indicating an amount of time that the genetic algorithm 510 has to execute before outputting a final output model, and the genetic algorithm 510 may determine a number of epochs that will be executed based on the specified time limit. To illustrate, an initial epoch of the genetic algorithm 510 may be timed (e.g., using a hardware or software timer at the computing device executing the genetic algorithm 510, and a total number of epochs that are to be executed within the specified time limit may be determined accordingly. As another example, the user may constrain a number of models evaluated in each epoch, for example by constraining the size of an input set 520 of models and/or an output set 530 of models.

In some implementations, a genetic algorithm 510 represents a recursive search process. Consequently, each iteration of the search process (also called an epoch or generation of the genetic algorithm 510 has an input set 520 of models (also referred to herein as an input population) and an output set 530 of models (also referred to herein as an output population). The input set 520 and the output set 530 may each include a plurality of models, where each model includes data representative of a machine learning data model. For example, each model may specify a neural network or an autoencoder by at least an architecture, a series of activation functions, and connection weights. The architecture, also referred to herein as a topology, of a model includes a configuration of layers or nodes and connections therebetween. The models may also be specified to include other parameters, including but not limited to bias values/functions and aggregation functions.

For example, each model can be represented by a set of parameters and a set of hyperparameters. In this context, the hyperparameters of a model define the architecture of the model (e.g., the specific arrangement of layers or nodes and connections), and the parameters of the model refer to values that are learned or updated during optimization training of the model. For example, the parameters include or correspond to connection weights and biases.

In a particular implementation, a model is represented as a set of nodes and connections therebetween. In such implementations, the hyperparameters of the model include the data descriptive of each of the nodes, such as an activation function of each node, an aggregation function of each node, and data describing node pairs linked by corresponding connections. The activation function of a node is a step function, sine function, continuous or piecewise linear function, sigmoid function, hyperbolic tangent function, or another type of mathematical function that represents a threshold at which the node is activated. The aggregation function is a mathematical function that combines (e.g., sum, product, etc.) input signals to the node. An output of the aggregation function may be used as input to the activation function.

In other implementations, the model is represented on a layer-by-layer basis. For example, the hyperparameters define layers, and each layer includes layer data, such as a layer type and a node count. Examples of layer types include fully connected, long short-term memory (LSTM) layers, gated recurrent units (GRU) layers, and convolutional neural network (CNN) layers. In some implementations, all of the nodes of a particular layer use the same activation function and aggregation function. In such implementations, specifying the layer type and node count fully may describe the hyperparameters of each layer. In other implementations, the activation function and aggregation function of the nodes of a particular layer can be specified independently of the layer type of the layer. For example, in such implementations, one fully connected layer can use a sigmoid activation function and another fully connected layer (having the same layer type as the first fully connected layer) can use a tanh activation function. In such implementations, the hyperparameters of a layer include layer type, node count, activation function, and aggregation function. Further, a complete autoencoder is specified by specifying an order of layers and the hyperparameters of each layer of the autoencoder.

In some implementations, a genetic algorithm 510 may be configured to perform speciation. For example, the genetic algorithm 510 may be configured to cluster the models of the input set 520 into species based on “genetic distance” between the models. The genetic distance between two models may be measured or evaluated based on differences in nodes, activation functions, aggregation functions, connections, connection weights, layers, layer types, latent-space layers, encoders, decoders, etc. of the two models. In an illustrative example, the genetic algorithm 510 may be configured to serialize a model into a bit string. In this example, the genetic distance between models may be represented by the number of differing bits in the bit strings corresponding to the models. The bit strings corresponding to models may be referred to as “encodings” of the models.

In some implementations, after configuration, a genetic algorithm 510 may begin execution based on the input data 502. Parameters of the genetic algorithm 510 may include but are not limited to, mutation parameter(s), a maximum number of epochs the genetic algorithm 510 will be executed, a termination condition (e.g., a threshold fitness value that results in termination of the genetic algorithm 510 even if the maximum number of generations has not been reached), whether parallelization of model testing or fitness evaluation is enabled, whether to evolve a feedforward or recurrent neural network, etc. As used herein, a “mutation parameter” affects the likelihood of a mutation operation occurring with respect to a candidate neural network, the extent of the mutation operation (e.g., how many bits, bytes, fields, characteristics, etc. change due to the mutation operation), and/or the type of the mutation operation (e.g., whether the mutation changes a node characteristic, a link characteristic, etc.). In some examples, the genetic algorithm 510 uses a single mutation parameter or set of mutation parameters for all of the models. In such examples, the mutation parameter may impact how often, how much, and/or what types of mutations can happen to any model of the genetic algorithm 510. In alternative examples, the genetic algorithm 510 maintains multiple mutation parameters or sets of mutation parameters, such as for individual or groups of models or species. In particular aspects, the mutation parameter(s) affect crossover and/or mutation operations, which are further described below.

In some implementations, for an initial epoch of a genetic algorithm 510, the topologies of the models in the input set 520 may be randomly or pseudo-randomly generated within constraints specified by the configuration settings or by one or more architectural parameters. Accordingly, the input set 520 may include models with multiple distinct topologies. For example, a first model of the initial epoch may have a first topology, including a first number of input nodes associated with a first set of data parameters, a first number of hidden layers including a first number and arrangement of hidden nodes, one or more output nodes, and a first set of interconnections between the nodes. In this example, a second model of the initial epoch may have a second topology, including a second number of input nodes associated with a second set of data parameters, a second number of hidden layers including a second number and arrangement of hidden nodes, one or more output nodes, and a second set of interconnections between the nodes. The first model and the second model may or may not have the same number of input nodes and/or output nodes. Further, one or more layers of the first model can be of a different layer type that one or more layers of the second model. For example, the first model can be a feedforward model, with no recurrent layers, whereas the second model can include one or more recurrent layers.

In some implementations, a genetic algorithm 510 may automatically assign an activation function, an aggregation function, a bias, connection weights, etc. to each model of the input set 520 for the initial epoch. In some aspects, the connection weights are initially assigned randomly or pseudo-randomly. In some implementations, a single activation function is used for each node of a particular model. For example, a sigmoid function may be used as the activation function of each node of the particular model. The single activation function may be selected based on configuration data. For example, the configuration data may indicate that a hyperbolic tangent activation function is to be used or that a sigmoid activation function is to be used. Alternatively, the activation function may be randomly or pseudo-randomly selected from a set of allowed activation functions, and different nodes or layers of a model may have different types of activation functions. Aggregation functions may similarly be randomly or pseudo-randomly assigned for the models in the input set 520 of the initial epoch. Thus, the models of the input set 520 of the initial epoch may have different topologies (which may include different input nodes corresponding to different input data fields if the data set includes many data fields) and different connection weights. Further, the models of the input set 520 of the initial epoch may include nodes having different activation functions, aggregation functions, and/or bias values/functions.

In some implementations, during execution, a genetic algorithm 510 performs fitness evaluation 540 and evolutionary operations 550 on the input set 520. In this context, fitness evaluation 540 includes evaluating each model of the input set 520 using a fitness function 542 to determine a fitness function value 544 (“FF values” in FIG. 5) for each model of the input set 520. The fitness function values 544 are used to select one or more models of the input set 520 to modify using one or more of the evolutionary operations 550. In FIG. 5, the evolutionary operations 550 include mutation operations 552, crossover operations 554, and extinction operations 556, each of which is described further below.

In some implementations, during a fitness evaluation 540, each model of the input set 520 is tested based on the input data 502 to determine a corresponding fitness function value 544. For example, a first portion 504 of the input data 502 may be provided as input data to each model, which processes the input data (according to the network topology, connection weights, activation function, etc., of the respective model) to generate output data. The output data of each model is evaluated using the fitness function 542 and the first portion 504 of the input data 502 to determine how well the model modeled the input data 502. In some examples, fitness of a model is based on reliability of the model, performance of the model, complexity (or sparsity) of the model, size of the latent space, or a combination thereof.

In some implementations, fitness evaluation 540 of the models of the input set 520 is performed in parallel. To illustrate, the system 500 may include devices, processors, cores, and/or threads 580 in addition to those that execute the genetic algorithm 510 and the optimization trainer 560. These additional devices, processors, cores, and/or threads 580 can perform the fitness evaluation 540 of the models of the input set 520 in parallel based on a first portion 504 of the input data 502 and may provide the resulting fitness function values 544 to the genetic algorithm 510.

In some implementations, a mutation operation 552 and a crossover operation 554 are highly stochastic under certain constraints and a defined set of probabilities optimized for model building, which produces reproduction operations that can be used to generate the output set 530, or at least a portion thereof, from the input set 520. In a particular implementation, the genetic algorithm 510 utilizes intra-species reproduction (as opposed to inter-species reproduction) in generating the output set 530. In other implementations, inter-species reproduction may be used in addition to or instead of intra-species reproduction to generate the output set 530. Generally, the mutation operation 552 and the crossover operation 554 are selectively performed on models that are more fit (e.g., have higher fitness function values 544), fitness function values 544 that have changed significantly between two or more epochs, or both).

In some implementations, an extinction operation 556 uses a stagnation criterion to determine when a species should be omitted from a population used as the input set 520 for a subsequent epoch of the genetic algorithm 510. Generally, the extinction operation 556 is selectively performed on models that are satisfy a stagnation criterion, such as modes that have low fitness function values 544, fitness function values 544 that have changed little over several epochs, or both.

In accordance with the present disclosure, cooperative execution of a genetic algorithm 510 and an optimization trainer 560 is used arrive at a solution faster than would occur by using a genetic algorithm 510 alone or an optimization trainer 560 alone. Additionally, in some implementations, the genetic algorithm 510 and the optimization trainer 560 evaluate fitness using different data sets, with different measures of fitness, or both, which can improve fidelity of operation of the final model. To facilitate cooperative execution, a model (referred to herein as a trainable model 532 in FIG. 5) is occasionally sent from the genetic algorithm 510 to the optimization trainer 560 for training. In a particular implementation, the trainable model 532 is based on crossing over and/or mutating the fittest models (based on the fitness evaluation 540) of the input set 520. In such implementations, the trainable model 532 is not merely a selected model of the input set 520; rather, the trainable model 532 represents a potential advancement with respect to the fittest models of the input set 520.

In some implementations, an optimization trainer 560 uses a second portion 506 of the input data 502 to train the connection weights and biases of the trainable model 532, thereby generating a trained model 562. The optimization trainer 560 does not modify the architecture of the trainable model 532.

In some implementations, during optimization, an optimization trainer 560 provides a second portion 506 of the input data 502 to the trainable model 532 to generate output data. The optimization trainer 560 performs a second fitness evaluation 570 by comparing the data input to the trainable model 532 to the output data from the trainable model 532 to determine a second fitness function value 574 based on a second fitness function 572. The second fitness function 572 is the same as the first fitness function 542 in some implementations and is different from the first fitness function 542 in other implementations. In some implementations, the optimization trainer 560 or portions thereof is executed on a different device, processor, core, and/or thread than the genetic algorithm 510. In such implementations, the genetic algorithm 510 can continue executing additional epoch(s) while the connection weights of the trainable model 532 are being trained by the optimization trainer 560. When training is complete, the trained model 562 is input back into (a subsequent epoch of) the genetic algorithm 510, so that the positively reinforced “genetic traits” of the trained model 562 are available to be inherited by other models in the genetic algorithm 510.

In implementations where the genetic algorithm 510 employs speciation, a species ID of each of the models may be set to a value corresponding to the species that the model has been clustered into. A species fitness may be determined for each of the species. The species fitness of a species may be a function of the fitness of one or more of the individual models in the species. As a simple illustrative example, the species fitness of a species may be the average of the fitness of the individual models in the species. As another example, the species fitness of a species may be equal to the fitness of the fittest or least fit individual model in the species. In alternative examples, other mathematical functions may be used to determine species fitness. The genetic algorithm 510 may maintain a data structure that tracks the fitness of each species across multiple epochs. Based on the species fitness, the genetic algorithm 510 may identify the “fittest” species, which may also be referred to as “elite species.” Different numbers of elite species may be identified in different embodiments.

In some implementations, a genetic algorithm 510 uses species fitness to determine if a species has become stagnant and is therefore to become extinct. As an illustrative non-limiting example, the stagnation criterion of the extinction operation 556 may indicate that a species has become stagnant if the fitness of that species remains within a particular range (e.g., +/−5%) for a particular number (e.g., 5) of epochs. If a species satisfies a stagnation criterion, the species and all underlying models may be removed from subsequent epochs of the genetic algorithm 510.

In some implementations, the fittest models of each “elite species” may be identified. The fittest models overall may also be identified. An “overall elite” need not be an “elite member,” e.g., may come from a non-elite species. Different numbers of “elite members” per species and “overall elites” may be identified in different embodiments.”

In some implementations, an output set 530 of the epoch is generated based on the input set 520 and the evolutionary operation 550. In the illustrated example, the output set 530 includes the same number of models as the input set 520. In some implementations, the output set 530 includes each of the “overall elite” models and each of the “elite member” models. Propagating the “overall elite” and “elite member” models to the next epoch may preserve the “genetic traits” resulted in caused such models being assigned high fitness values.

In some implementations, the rest of the output set 530 may be filled out by random reproduction using the crossover operation 554 and/or the mutation operation 552. After the output set 530 is generated, the output set 530 may be provided as the input set 520 for the next epoch of the genetic algorithm 510.

In some implementations, after one or more epochs of a genetic algorithm 510 and one or more rounds of optimization by an optimization trainer 560, the system 500 selects a particular model or a set of models as the final model (e.g., a model that is executable to perform one or more of the model-based operations of FIGS. 1-4B). For example, the final model may be selected based on the fitness function values 544, 574. For example, a model or set of models having the highest fitness function value 544 or 574 may be selected as the final model. When multiple models are selected (e.g., an entire species is selected), an ensemble can be generated (e.g., based on heuristic rules or using the genetic algorithm 510 to aggregate the multiple models. In some implementations, the final model can be provided to the optimization trainer 560 for one or more rounds of optimization after the final model is selected. Subsequently, the final model can be output for use with respect to other data (e.g., real-time data).

Although the examples described above are largely described in the context of a single ventilator whose operation may be controlled and monitored by one or more trained models, in other embodiments the techniques described herein may be applied across a fleet of ventilators. In such an embodiment, a centralized resource may be communicatively coupled (directly or indirectly) to each of the ventilators that the centralized resource is responsible for controlling and monitoring. The centralized resource may be embodied, for example, as one or more modules of computer program instructions executing on physical or virtualized computer hardware. For example, the centralized resource may be embodied as one or more computing systems that are supporting the execution of a predictive maintenance product such as SparkPredict™ from SparkCognition™.

In such an example, each of the ventilators may be equipped with one or more sensors as described above, and sensor data may be gathered and ultimately communicated to the centralized resource. Upon receiving the sensor data from the ventilators, the centralized resource may use sensor data (as well as other data related to the ventilators such as age, amount of time in use, information describing specific components of each ventilator, and more) to evaluate the health of the ventilator, to generate alerts that some maintenance event is predicted to be needed, and so on. Such sensors can include not only the sensors described above, but may also include additional sensors that are coupled to the ventilator itself, such as a temperature sensor to monitor the temperature of various components with the ventilator, a vibration sensor to monitor vibrations within the sensor, and other sensors as will occur to those of skill in the art. As such, failures or problems may be predicted before the actual occurrence of the failure or problem, such that remedial actions can be taken prior to the occurrence of an event that would cause the ventilator to cease operating as desired.

Readers will appreciate that in order to generate predictive maintenance models, training may take place to classify operating states or learning what is “normal behavior” of the ventilator based on sensor output, such that the models can flag anomalies or flag other deviations from normal behavior. When an alert is generated by these models, a review can be conducted to identify which features (i.e., sensors) contributed most to the alert and a determination may be made as to whether a failure actually occurred, or an anomalous state was entered into. In some examples, the features contributing most to the alert may be automatically determined using relative feature importance estimation or other techniques. Furthermore, a label may be provided for the failure/state and a decision may be reached as to whether the models should be trained to identity that condition in the future (in which case the condition could be identified with even more lead time by virtue of having included in the training data a time series of sensor values leading up to the alert). In addition, if there are specific solution steps that worked to fix the problem, those solution steps could be linked to this now predictable condition, thereby enabling not just predictive maintenance but also prescriptive maintenance. Additional information related to the models and workflows that can enable predictive maintenance and prescriptive maintenance may be found in U.S. Ser. No. 63/014,678 (hereafter, ‘the parent application’), all of which is incorporated by reference into the present disclosure.

In some embodiments, the ability to perform predictive maintenance may be developed, at least in part, through the usage of one or more trained autoencoders. The one or more trained autoencoders may be generated and trained in a variety of ways, include through the use of automated model building systems and methods that cooperatively use a genetic algorithm and selective optimization training to generate and train an autoencoder to monitor one or more devices for anomalous operational states, as described in greater detail in the parent application. In such embodiments, combining a genetic algorithm with selective optimization training as enables generation of an autoencoder that is able to detect anomalous operation significantly before failure of a monitored device (in this case a ventilator), where the cooperative use of the genetic algorithms and optimization training is faster and uses fewer computing resources than training a monitoring autoencoder using backpropagation or genetic algorithms alone, although those individual techniques may also be used alone in some embodiments. Such autoencoders may have symmetric or asymmetric topologies, as further described herein and in the parent application.

In accordance with the described techniques, a combination of a genetic algorithm and an optimization algorithm (such as backpropagation, a derivative free optimizer (DFO), an extreme learning machine (ELM), or a similar optimizer) may be used to generate and then train an autoencoder. As used herein, the terms “optimizer,” “optimization trainer,” and similar terminology, is not to be interpreted as requiring such components or steps to generate literal optimal results (e.g., 100% prediction or classification accuracy). Rather, these terms indicate an attempt to generate an output that is improved in some fashion relative to an input. For example, an optimization trainer that receives a trainable autoencoder as input and outputs a trained autoencoder may attempt to decrease reconstruction losses of the trainable autoencoder by modifying one or more attributes of the trainable autoencoder.

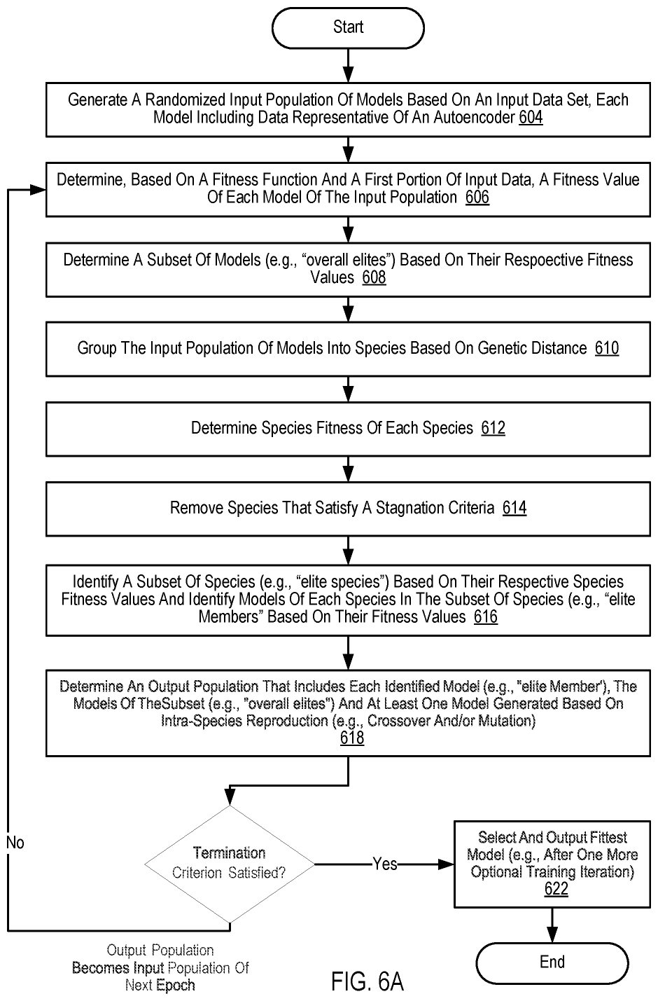

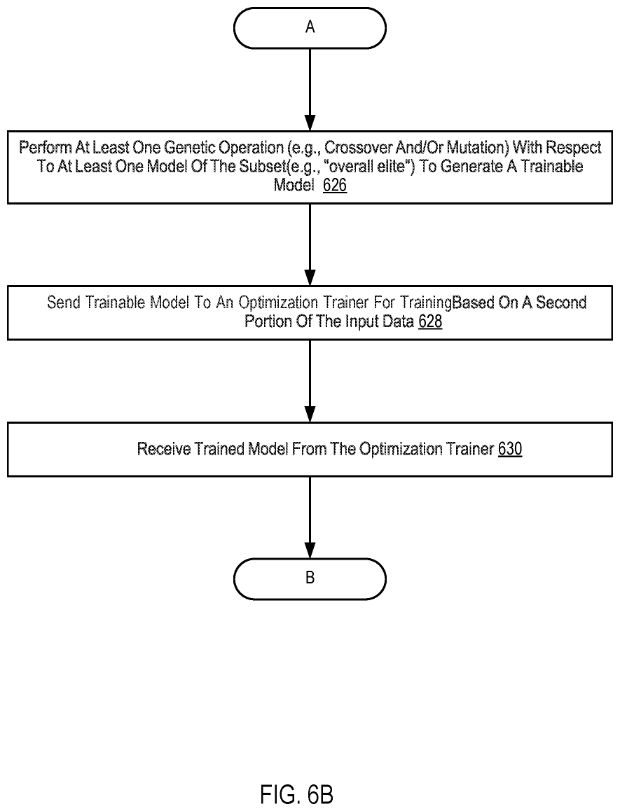

For further explanation, FIG. 6A and FIG. 6B collectively illustrate an example of a method of cooperative execution of a genetic algorithm and an optimization trainer to generate an autoencoder in accordance with some embodiments of the present disclosure. The example method depicted here may be performed in a system as described in greater detail in the parent application. The method 600 may start, at 602, and may include generating a randomized input population of models based on an input data set, at 604. Each model may include data representative of an autoencoder and each of the models may be part of an input set.

The method 600 may also include determining, based on a fitness function, a fitness value of each model of the input population, at 606. For example, the fitness of each model of the input set may be determined based on the first portion of the input data. The method 600 may further include determining a subset of models based on their respective fitness values, at 608. The subset of models may be the fittest models of the input population, e.g., “overall elites.” For example, “overall elites” may be determined as described with reference to the parent application.

The method 600 may also include performing multiple sets of operations at least partially concurrently. Continuing to 626 (in FIG. 6B), the method 600 may include performing at least one genetic operation with respect to at least one model of the subset to generate a trainable autoencoder. For example, the crossover operation, the mutation operation, or both, may be performed with respect to the “overall elites” to generate the trainable autoencoder.

The method 600 may also include sending the trainable model to an optimization trainer (e.g., a backpropagation trainer) for training based on a second portion of the input data, at 628. For example, an optimization trainer may train the trainable autoencoder based on the second portion of the input data to generate the trained autoencoder, as described with in the parent application.

Returning to FIG. 6A, the genetic algorithm may continue while optimization training occurs. For example, the method 600 may include grouping the input population of models into species based on genetic distance, at 610, and determining species fitness of each species, at 612. To illustrate, the models of the input set may be grouped into species and species fitness may be evaluated as described in the parent application.

Continuing to 614, species that satisfy a stagnation criterion may be removed. At 616, the method 600 may include identifying a subset of species based on their respective fitness values and identifying models of each species in the subset based on their respective model fitness values. The subset of species may be the fittest species of the input population, e.g., “elite species,” and the identified models of the “elite species” may be the fittest members of those species, e.g., “elite members.” For example, species fitness values, “elite species,” and “elite members” may be determined.

The method 600 may include determining an output population that includes each “elite member,” the “overall elites,” and at least one model that is generated based on intra-species reproduction, at 618. For example, the models of the output set may be determined, where the output set includes the overall elite models, the elite members (including the elite member model), and at least one model generated based on intra-species reproduction using the crossover operation and/or the mutation operation.

The method 600 may include determining whether a termination criterion is satisfied, at 620. The termination criterion may include a time limit, a number of epochs, or a threshold fitness value of an overall fittest model, as illustrative non-limiting examples. If the termination criterion is not satisfied, the method 600 returns to 606 and a next epoch of the genetic algorithm is executed, where the output population determined at 618 is the input population of the next epoch. As described herein, while the genetic algorithm is ongoing, the optimization trainer may train the trainable autoencoder to generate a trained autoencoder. When training is complete, the method 600 may include receiving the trained autoencoder from the optimization trainer, at 630 (in FIG. 6B). The trained autoencoder may be added to the input set of an epoch of the genetic algorithm, as shown in FIG. 6B. When the termination criterion is satisfied, at 620, the method 600 may include selecting and outputting a fittest model, at 622, and the method 600 may end, at 624. In some implementations, the selected model may be subjected to a final training operation, e.g., by the optimization trainer or by another trainer, before being output.

It is to be understood that the division and ordering of steps in FIGS. 6A and 6B is for illustrative purposes only and is not to be considered limiting. In alternative implementations, certain steps may be combined and other steps may be subdivided into multiple steps. Moreover, the ordering of steps may change. For example, the termination criterion may be evaluated after determining the “overall elites,” at 608, rather than after determining the output population, at 618.

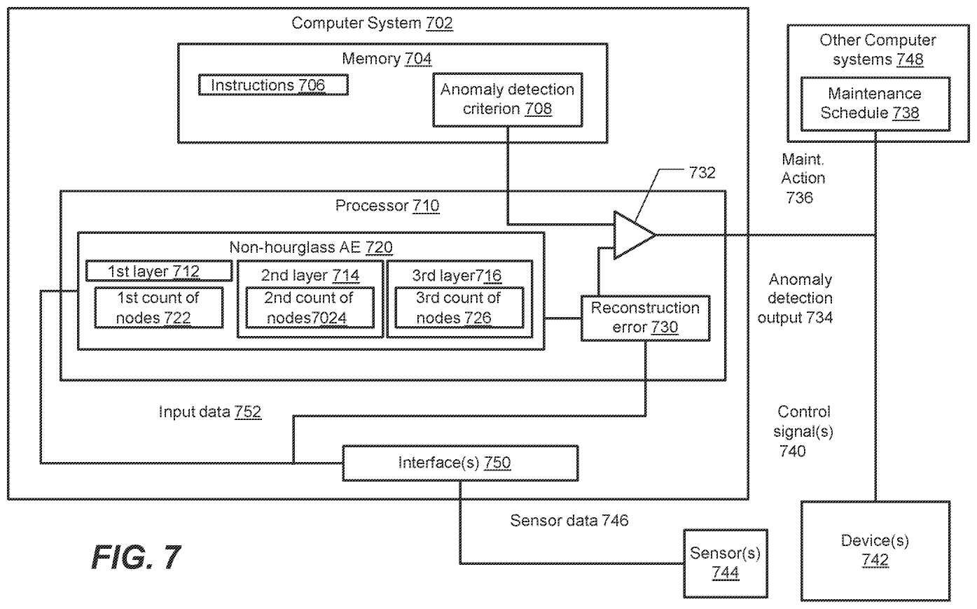

For further explanation, FIG. 7 illustrate an example of a system 700 for using an autoencoder to monitor one or more devices for operational anomalies in accordance with some embodiments of the present disclosure. In this particular example the system 700 uses a non-hourglass autoencoder 720, although other autoencoders such as a non-mirrored autoencoder may also be used. In some implementations, the non-hourglass autoencoder 720, a non-mirrored autoencoder, or both, are generated using the system described in the parent application.

The system 700 includes a computer system 702 including a processor 710 and memory 704. The memory 704 stores instructions 706 that are executable by the processor 710 to monitor sensor data 746 from one or more devices 742 and to generate an anomaly detection output 734 based on the sensor data 746.

The computer system 702 also includes one or more interface devices 750 which are coupled to one or more sensors 744, the device(s) 742, one or more other computer devices (e.g., a computing device that stores a maintenance schedule 738), or a combination thereof. In the example illustrated here, the interface(s) 750 receive the sensor data 746 (in real-time or delayed) from the sensor(s) 744. The sensor data 746 represents operational states of the device(s) 742. For example, the sensor data 746 can include temperature data, pressure data, rotation rate data, vibration data, power level data, or any other data that is indicative of the behavior or operational state of the device(s) 742.

The interface(s) 750 or the processor 710 generate input data 752 based on the sensor data 746. For example, the input data 752 can be identical to the sensor data 746, of the input data 752 can be generated by modifying the sensor data 746. Examples of modifications that can be used to convert the sensor data 746 into the input data 752 include filling in missing data (e.g., by extrapolation), dropping some data, smoothing data, time synchronizing two or more sets of data that have different time signatures or sampling intervals, combining data with other information to generate new data (e.g., calculating a power value based on measured current and voltage values), etc.

The processor 710 provides the input data 752 to an autoencoder (e.g., the non-hourglass autoencoder or other form of autoencoder). An encoder portion of the autoencoder 720 dimensionally reduces the input data 752 to a latent space. A decoder portion of the autoencoder 720 then attempts to recreate the input data using the dimensionally reduced data. Output data from the decoder side is compared to the input data 752 to determine reconstruction error 730. A comparator 732 compares the reconstruction error 730 to an anomaly detection criterion 708 to determine whether the sensor data 746 corresponds to an anomalous or abnormal operational state of the device(s) 742. To illustrate, the autoencoder is 720 may be trained to detect deviation from “normal” behavior, and such deviation may correspond to relatively large amounts of reconstruction error 730. The comparator 732 generates the anomaly detection output 734 when the reconstruction error 730 satisfies the anomaly detection criterion 708. In a particular example, the anomaly detection criterion 708 is satisfied when the reconstruction error 730 is greater than a threshold specified by the anomaly detection criterion 708. In another particular example, the anomaly detection criterion 708 is satisfied when an aggregate or average value of the reconstruction error 730 is greater than a threshold specified by the anomaly detection criterion 708. Other anomaly detection criteria 708 can also, or in the alternative, be used.

Generally, the reconstruction error 730 is relatively low in response to the sensor data 746 representing normal operational states of the device(s) 742 and is relatively high in response to the sensor data 746 representing anomalous or abnormal operational states of the device(s) 742. For example, when the autoencoder 720 is generated using the system 700, the cooperative use of the genetic algorithm with the first portion of the input data and the optimization trainer with the second portion of the input data ensures that the autoencoder 720 has a wide range for reconstruction errors 730 depending on whether the sensor data 746 represent normal or abnormal operational states of the device(s) 742.

The anomaly detection output 734 can be sent to another device, such one or more other computers systems 748, to the device(s) 742, etc. For example, when the other computer system(s) 748 stores or maintains a maintenance schedule 738 for the device(s) 742, the anomaly detection output 734 can include or correspond to a maintenance action instruction 736 that causes the other computer system(s) 748 to update the maintenance schedule 738 to schedule maintenance for the device(s) 742. As another example, the anomaly detection output 734 can include or correspond to a control signal 740 that is provided to the device(s) 742 or to a control or control system associated with the device(s) 742 to change operation of the device(s) 742. For example, the control signal 740 may cause the device(s) 742 to shut down, to change one or more operational parameters, to restart or reset, etc.

In FIG. 7, the non-hourglass autoencoder 720 includes a plurality of layers, including a first layer 712 having a first count of nodes 722, a second layer 714 having a second count of nodes 724, and a third layer 716 having a third count of nodes 726. The first, second, and third layers 712, 714, 716 are adjacent to one another in either the encoder portion of the non-hourglass autoencoder 720 or the decoder portion of the non-hourglass autoencoder 720. If the first, second, and third layers 712, 714, 716 are in the encoder portion of the nonhourglass autoencoder 720, the second count of nodes 724 is greater than the first count of nodes 722. For example, as illustrated in the parent application, the first layer 712 may correspond to the input layer, the second layer 714 may correspond to the first hidden layer, and the third layer 716 may correspond to the second hidden layer. If the first, second, and third layers 712, 1214, 716 are in the decoder portion of the non-hourglass autoencoder 720, the second count of nodes 724 is greater than the third count of nodes 726.

Thus, the second layer 714 is a hidden layer that has more nodes than would be present in an hourglass architecture. The additional nodes of this hidden layer provide useful convolution of the data being processed which improves discrimination between sensor data 746 representing normal and abnormal operational states of the device(s) 742. For example, the additional data convolution provided by the second layer 714 may decrease reconstruction error 730 associated with sensor data 746 representing normal operational states of the device(s) 742, may increase reconstruction error 730 associated with sensor data 746 representing abnormal operational states of the device(s) 742, or both.

In the examples described herein, sensor values may be monitored not only for the purposes of predicting that maintenance needs to be performed or identifying solution steps that may be taken, but sensor values may also be monitored for adherence to regulatory rules, best practices, or some predetermined operational requirement. For example, sensor values may be used to determine whether some component (e.g., the manual resuscitator, the motor) are operating within predetermined device specifications.

Readers will further appreciate that while predictive maintenance operations may be performed by the centralized resource, other forms of monitoring or controlling the ventilators may also be performed by the same centralized resource or by another centralized resource. For example, a centralized resource may be responsible for receiving sensor data related to the health of the patient and may make recommendations or actually change the operation of a ventilator based on the sensor data. For example, if the set of sensors includes an oximeter and data from the oximeter indicates that the oxygen saturation of a patient's blood is below a desired level, the centralized resource may generate an alert that is displayed on the ventilator itself, sent to a medical professional, or otherwise distributed. Alternatively, the centralized resource may be configured to adjust the operation of the ventilator (e.g., adjusting the operating to increase the tidal volume) in an effort to improve the patient's condition.

Although the embodiments described above largely relate to embodiments where the operation of a ventilator is monitored and controlled, in other embodiments the techniques described above may be applied to other medical devices. In fact, any medical devices whose operation may be monitored through the usage of sensors or other device that is capable of quantifying aspects related to a medical device's operation may also be monitored and controlled in a similar fashion. Such medical devices may include, as a non-limiting list of examples, anesthetic devices, dialysis machines, sleep diagnostic devices, sleep apnea therapy devices, infusion pumps, oxygen concentrators, and many others.

The systems and methods illustrated herein may be described in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, Java, JavaScript, VBScript, Macromedia Cold Fusion, COBOL, Microsoft Active Server Pages, assembly, PERL, PHP, AWK, Python, Visual Basic, SQL Stored Procedures, PL/SQL, any UNIX shell script, and extensible markup language (XML) with the various algorithms being implemented with any combination of data structures, objects, processes, routines or other programming elements. Further, it should be noted that the system may employ any number of techniques for data transmission, signaling, data processing, network control, and the like.

The systems and methods of the present disclosure may be embodied as a customization of an existing system, an add-on product, a processing apparatus executing upgraded software, a standalone system, a distributed system, a method, a data processing system, a device for data processing, and/or a computer program product. Accordingly, any portion of the system or a module or a decision model may take the form of a processing apparatus executing code, an internet based (e.g., cloud computing) embodiment, an entirely hardware embodiment, or an embodiment combining aspects of the internet, software, and hardware. Furthermore, the system may take the form of a computer program product on a computer-readable storage medium or device having computer-readable program code (e.g., instructions) embodied or stored in the storage medium or device. Any suitable computer-readable storage medium or device may be utilized, including hard disks, CD-ROM, optical storage devices, magnetic storage devices, and/or other storage media. As used herein, a “computer-readable storage medium” or “computer-readable storage device” is not a signal.

Systems and methods may be described herein with reference to screen shots, block diagrams and flowchart illustrations of methods, apparatuses (e.g., systems), and computer media according to various aspects. It will be understood that each functional block of a block diagrams and flowchart illustration, and combinations of functional blocks in block diagrams and flowchart illustrations, respectively, can be implemented by computer program instructions.

Computer program instructions may be loaded onto a computer or other programmable data processing apparatus to produce a machine, such that the instructions that execute on the computer or other programmable data processing apparatus create means for implementing the functions specified in the flowchart block or blocks. These computer program instructions may also be stored in a computer-readable memory or device that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

Although the disclosure may include a method, it is contemplated that it may be embodied as computer program instructions on a tangible computer-readable medium, such as a magnetic or optical memory or a magnetic or optical disk/disc. All structural, chemical, and functional equivalents to the elements of the above-described exemplary embodiments that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

In this written description, common features are designated by common reference numbers throughout the drawings. As used herein, various terminology is used for the purpose of describing particular implementations only and is not intended to be limiting. For example, the singular forms “a,” “an,” and “the” are intended to include the plural forms as well, unless the context clearly indicates otherwise. It may be further understood that the terms “comprise,” “comprises,” and “comprising” may be used interchangeably with “include,” “includes,” or “including.” Additionally, it will be understood that the term “wherein” may be used interchangeably with “where.” As used herein, “exemplary” may indicate an example, an implementation, and/or an aspect, and should not be construed as limiting or as indicating a preference or a preferred implementation. As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). As used herein, the term “set” refers to a grouping of one or more elements, and the term “plurality” refers to multiple elements. In the present disclosure, terms such as “determining,” “calculating,” “estimating,” “shifting,” “adjusting,” etc. may be used to describe how one or more operations are performed. It should be noted that such terms are not to be construed as limiting and other techniques may be utilized to perform similar operations. Additionally, as referred to herein, “generating,” “calculating,” “estimating,” “using,” “selecting,” “accessing,” and “determining” may be used interchangeably. For example, “generating,” “calculating,” “estimating,” or “determining” a parameter (or a signal) may refer to actively generating, estimating, calculating, or determining the parameter (or the signal) or may refer to using, selecting, or accessing the parameter (or signal) that is already generated, such as by another component or device. As used herein, “coupled” may include “communicatively coupled,” “electrically coupled,” or “physically coupled,” and may also (or alternatively) include any combinations thereof. Two devices (or components) may be coupled (e.g., communicatively coupled, electrically coupled, or physically coupled) directly or indirectly via one or more other devices, components, wires, buses, networks (e.g., a wired network, a wireless network, or a combination thereof), etc. Two devices (or components) that are electrically coupled may be included in the same device or in different devices and may be connected via electronics, one or more connectors, or inductive coupling, as illustrative, non-limiting examples. In some implementations, two devices (or components) that are communicatively coupled, such as in electrical communication, may send and receive electrical signals (digital signals or analog signals) directly or indirectly, such as via one or more wires, buses, networks, etc. As used herein, “directly coupled” may include two devices that are coupled (e.g., communicatively coupled, electrically coupled, or physically coupled) without intervening components.

Changes and modifications may be made to the disclosed embodiments without departing from the scope of the present disclosure. These and other changes or modifications are intended to be included within the scope of the present disclosure, as expressed in the following claims.

What is claimed is:

1. A ventilator, comprising:
a manual resuscitator;
a motor; and
a compression plate that is mechanically coupled to the motor, wherein operation of the motor causes the compression plate to periodically compress the manual resuscitator against a resuscitator plate.

2. The ventilator of claim 1 further comprising one or more operational parameter adjustment controls, wherein operation of the motor is adjusted in response to a value of a particular operational parameter being changed using the one or more operational parameter adjustment controls.

3. The ventilator of claim 1 wherein the compression plate is mechanically coupled to the motor by a movable linkage system.

4. The ventilator of claim 1 further comprising one or more sensors selected from the group consisting of a pressure sensor, a patient pulse oximeter, an inline oxygen flow meter, a mass flow sensor, an electrical sensor, a humidity sensor, a temperature sensor, and combinations thereof.

5. The ventilator of claim 4 further comprising one or more computer processors, wherein the one or more computer processors execute computer program instructions that adjust the operation of the ventilator in response to sensor data received from the one or more sensors.

6. The ventilator of claim 4 further comprising one or more computer processors, wherein the one or more computer processors execute computer program instructions that generate alerts in response to sensor data received from the one or more sensors.

7. A method of operating a ventilator, the method comprising:
operating an electric motor, the electric motor mechanically coupled to a compression plate, wherein operation of the electric motor causes the compression plate to periodically compress a manual resuscitator against a resuscitator plate; and
responsive to receiving a signal, adjusting the operation of the electric motor.

8. The method of claim 7 wherein the signal is received in response to a value of a particular operational parameter being changed using one or more operational parameter adjustment controls.

9. The method of claim 7 wherein the ventilator includes one or more sensors selected from the group consisting of a pressure sensor, a patient pulse oximeter, an inline oxygen flow meter, a mass flow sensor, an electrical sensor, a humidity sensor, a temperature sensor, and combinations thereof.

10. The method of claim 9 further comprising:
receiving, from the one or more sensors, sensor data corresponding to a condition of one or more components of the ventilator; and
determining, using a trained model, whether the one or more components of the ventilator are operating in an acceptable manner.

11. The method of claim 10 further comprising, responsive to determining that the one or more components of the ventilator are not operating in an acceptable manner, generating a signal to adjust operation of the electric motor.

12. The method of claim 10 further comprising, responsive to determining that the one or more components of the ventilator are not operating in an acceptable manner, generating an alert.

13. The method of claim 12 wherein the alert includes information describing a projected failure of one or more components of the ventilator.

14. The method of claim 7 further comprising determining, using a trained model, one or more initial operating parameters for the ventilator based on information describing a patient.

15. A computer program product for operating a ventilator, the computer program product disposed on a non-transitory computer readable medium, the computer program product includes computer program instructions that, when executed by a computer processor, cause the processor to perform the steps of:
operating a motor, the motor mechanically coupled to a compression plate, wherein operation of the motor causes the compression plate to periodically compress a manual resuscitator against a resuscitator plate; and
responsive to receiving a signal, adjusting the operation of the motor.

16. The computer program product of claim 15 wherein the signal is received in response to a value of a particular operational parameter being changed using one or more operational parameter adjustment controls.

17. The computer program product of claim 15 further comprising computer program instructions that, when executed by a computer processor, cause the processor to perform the steps of:
receiving, from the one or more sensors, sensor data corresponding to a condition of one or more components of the ventilator; and
determining, using a trained model, whether the one or more components of the ventilator are operating in an acceptable manner.

18. The computer program product of claim 17 further comprising computer program instructions that, when executed by a computer processor, cause the processor to perform the step of, responsive to determining that the one or more components of the ventilator are not operating in an acceptable manner, generating a signal to adjust operation of the motor.

19. The computer program product of claim 17 further comprising computer program instructions that, when executed by a computer processor, cause the processor to perform the step of, responsive to determining that the one or more components of the ventilator are not operating in an acceptable manner, generating an alert.

20. The computer program product of claim 19 wherein the alert includes information describing a projected failure of one or more components of the ventilator.
